(19) United States
(12) Patent Application Publication
(10) Pub. No.: US 2024/0319196 A1
(43) Pub. Date: Sep. 26, 2024

(54) METHODS FOR PREDICTING MULTI-ORGAN METASTATIC DISEASE AND OVERALL AND PROGRESSION FREE SURVIVAL IN SUBJECTS HAVING HYPER-ENGORGED CIRCULATING CANCER ASSOCIATED MACROPHAGE-LIKE CELLS (CAMLS)

(71) Applicant: Creatv Micro Tech Inc., Potomac, MD (US)

(72) Inventors: Daniel L. Adams, Basking Ridge, NJ (US); Cha-Mei TANG, Potomac, MD (US)

(73) Assignee: Creatv Micro Tech Inc., Potomac, MD (US)

(21) Appl. No.: 18/576,514

(22) PCT Filed: Jul. 6, 2022
(86) PCT No.: PCT/US22/36253

§ 371 Date: Jan. 4, 2024

Related U.S. Application Data
(60) Provisional application No. 63/218,628, filed on Jul. 6, 2021.

(51) Int. Cl. G01N 33/574; G01N 33/569; G01N 33/68

(52) U.S. Cl. CPC G01N 33/57492; G01N 33/56972; G01N 33/6872

(57) ABSTRACT

Means for predicting (i) multiple organ metastasis and/or multifocal metastatic disease and (ii) overall survival (OS) and progression free survival (PFS) of subjects having cancer are disclosed, where the predictions are based on the number and size of circulating cancer associated macrophage-like cells (CAMLs) found in a biological sample, such as blood, from the subject.

### BACKGROUND

### Field of the Invention

The present invention generally relates to the use of biomarkers in the blood and other bodily fluids to make diagnoses regarding cancer and predictions regarding overall survival and progression free survival in subjects having cancer, such as multifocal metastatic disease.

### Related Art

When tumor cells break away from primary solid tumors, they penetrate into the blood or lymphatic circulation, and ultimately leave the blood stream and enter either organs or tissue to form metastasis. 90% of cancer-related deaths are caused by the metastatic process. The most common metastatic sites are the lung, liver, bone and brain. Tumor cells found in the circulation are called circulating tumor cells (CTCs). Many research publications and clinical trials show that CTCs have clinical utility in (i) providing prognostic survival and cancer recurrence information through the enumeration of CTCs in the blood stream, and (ii) providing treatment information through examination of protein expression levels, and the occurrence of gene mutations and translocations in the CTCs. However, CTCs are not consistently associated with the development and/or presence of cancer in a subject, even in stage IV cancer patients. While CTCs are found most often in small cell lung cancer and stage IV of breast, prostate and colorectal cancers, they are rare in early stages of the same cancer. CTCs are also rare in other cancers.

Circulating cancer associated macrophage-like cells (CAMLs) are another cancer-related cell type that is found in the blood of subjects having cancer. CAMLs are associated with all solid tumors tested and all stages of cancer CAMLs are polyploid and very large in size, ˜25 μm to ≥300 μm in size. These polyploid cells can be either CD45(−) or CD45(+), and can express CD11c, CD14 and CD31, which confirms their origin as a myeloid lineage. They are often found in the process of engulfing CTCs and cellular debris [1,7].

Identifying predictive biomarkers that can differentiate between aggressive and non-aggressive metastases remain elusive in the field of precision oncology [11]. Due to significantly worsened survival associated with multi-organ metastases, there is a need for biomarkers that can predict for these patient subpopulations [10,11]. Prior prospective research on CAMLs has suggested use of these cells as a universal, minimally invasive blood-based prognostic & predictive biomarker in multiple solid malignancies [1,9].

Assays associated with the identification and characterization of biomarkers, such as CAMLs, in blood and other body fluids can be used to provide prognostic information. The present invention is directed to providing such tools to clinicians and other important goals.

### SUMMARY

The present invention is directed methods of using a type of cell with unique characteristics that is found in the blood of subjects having solid tumors, including carcinoma, sarcoma, neuroblastoma and melanoma. These circulating cells, termed “Cancer Associated Macrophage-like cells” (CAMLs), have been shown to be associated with the presence of solid tumors in a subject having cancer. Five morphologies associated with CAMLs have been characterized and described [1,2]. CAMLs are a circulating stromal cell subtype that have been found consistently in the peripheral blood of subjects having stage I to stage IV solid tumors by microfiltration based on size exclusion using microfilters with precision pore size [1].

Medical applications associated with CAMLs include, but are not limited to, use of the cells as a biomarker to provide early detection of cancer and diagnosis of cancer, in particular, in the early detection and diagnosis of cancer relapse or recurrence, and in the determination of cancer mutation. CAMLs have also been shown to have clinical utility as a biomarker in making predictions regarding disease progression and patient survival.

Patients with multiple organ metastases have poorer prognoses and higher tumor burden than those with single organ metastasis [1]. While numerous studies have shown that CAMLs having a size≥50 μm predict poor clinical outcomes, meta-analysis of these studies now suggests that hyper-engorged CAMLs (heCAMLs), i.e cells having a size of about ≥100 μm, are associated with multifocal metastatic disease, as well as non-metastatic disease, and even worse outcomes [9]. As shown in the experimental evidence provided herein, the presence of heCAMLs in patients with cancer is correlated with multi-organ spread and shorter Progression Free Survival (PFS) and Overall Survival (OS).

In a first embodiment, the present invention is directed to methods for diagnosing multiple organ metastasis and/or multifocal metastatic disease in a subject. The methods comprise determining the size of CAMLs in a biological sample from a subject, such as a subject having cancer, wherein when at least one CAML in said sample is about 100 μm or more in size, the subject is diagnosed to have multiple organ metastases and/or multifocal metastatic disease.

In a second embodiment, the present invention is directed to methods for predicting development of multiple organ metastasis and/or multifocal metastatic disease in a subject. The methods comprise determining the size of CAMLs in a biological sample from a subject, such as a subject having cancer, wherein when at least one CAML in said sample is about 100 μm or more in size, the subject is predicted to develop multiple organ metastases and/or multifocal metastatic disease.

In a third embodiment, the invention is directed to methods for predicting overall survival (OS) and/or progression free survival (PFS) of a subject having cancer based on CAML cell size. The methods comprise determining the size of CAMLs in a biological sample from a subject, such as a subject having cancer, wherein when at least one CAML in said sample is about 100 μm or more in size, the subject is predicted to have shorter OS and/or shorter PFS than a subject having the same or similar cancer but lacking at least one CAML in a corresponding sample about 100 μm or more in size. Thus, the OS and/or PFS of a subject with larger-sized CAMLs is predicted to be less or shorter than the OS and/or PFS of a subject having smaller-sized CAMLs. In certain aspects, the cancer is multiple organ metastasis or multifocal metastatic disease.

In one aspect, the method comprises determining the size of CAMLs in a biological sample from a subject having cancer, wherein when at least one CAML in said sample is about 100 μm or more in size, the OS and/or PFS of the subject is predicted to be less or shorter than a subject having cancer where none of the CAMLs is more than about 100 μm in size.

In certain aspects of the embodiments of the invention, OS or PFS, or both, is over a period of at least 12 months. In other aspects of the embodiments of the invention, OS or PFS, or both, is over a period of at least 24 months.

In certain aspects of the embodiments of the invention, the size of the biological sample is between 5 and 15 mL.

In each of the embodiments and aspects of the invention, the CAMLs can be defined as having each of the following characteristics:
(a) large atypical polyploid nucleus of about 14-64 μm in size, or multiple nuclei in a single cell;
(b) cell size of about 20-300 μm in size; and
(c) morphological shape selected from the group consisting of spindle, tadpole, round, oblong, two legs, more than two legs, thin legs, and amorphous.

In certain aspects of the embodiments of the invention, the CAMLs can be further defined as having one or more of the following additional characteristics:
(d) CD14 positive phenotype;
(e) CD45 expression;
(f) EpCAM expression;
(g) vimentin expression;
(h) PD-L1 expression;
(i) monocytic CD11C marker expression;
(j) endothelial CD146 marker expression;
(k) endothelial CD202b marker expression, and
(l) endothelial CD31 marker expression.

In each aspect and embodiment of the invention, the CAMLs larger than about 100 μm are termed “hyper-engorged Cancer Associated Macrophage-like cells” or heCAMLs.

In certain aspects of the embodiments of the invention, the source of the biological sample may be, but is not limited to, one or more of peripheral blood, blood, lymph node, bone marrow, cerebral spinal fluid, tissue, and urine. When the biological sample is blood, the blood may be antecubital-vein blood, inferior-vena-cava blood, femoral vein blood, portal vein blood, or jugular-vein blood, for example. The sample may be a fresh sample or a properly prepared cryo-preserved sample that is thawed.

In certain aspects of the embodiments of the invention, the cancer is a solid tumor, Stage I cancer, Stage II cancer, Stage III cancer, Stage IV cancer, carcinoma, sarcoma, neuroblastoma, melanoma, epithelial cell cancer, breast cancer, prostate cancer, lung cancer, pancreatic cancer, colorectal cancer, renal cancer, liver cancer, head and neck cancer, kidney cancer, ovarian cancer, esophageal cancer or other solid tumor cancer.

In certain aspects of the embodiments of the invention, the circulating cells are isolated from the biological samples for the determining steps using one or more means selected from size exclusion methodology, immunocapture, red blood cell lysis, white blood cell depletion, FICOLL, electrophoresis, dielectrophoresis, flow cytometry, magnetic levitation, and various microfluidic chips, or a combination thereof.

In one aspect of the invention, circulating cells are isolated from the biological samples using size exclusion methodology that comprises using a microfilter. The microfilter may have a pore size ranging from about 5 microns to about 20 microns. The pores of the microfilter may have a round, race-track shape, oval, square and/or rectangular pore shape. The microfilter may have precision pore geometry and/or uniform pore distribution.

In another aspect of the invention, circulating cells are isolated from the biological samples using a microfluidic chip via physical size-based sorting, hydrodynamic size-based sorting, grouping, trapping, immunocapture, concentrating large cells, or eliminating small cells based on size.

In a further aspect of the invention, circulating cells are isolated from the biological samples using a CellSieve™ low-pressure microfiltration assay.

### BRIEF DESCRIPTION OF THE DRAWINGS

FIG. 1 shows the intensity of cell differentiation markers used to identify and subtype CAMLs.

FIG. 2 shows the distribution of heCAMLs in biological samples from (A) single organ and (B) multi-organ metastatic patients via four cancer types: breast, lung, prostate and renal.

FIG. 3 shows Kaplan-Meier comparisons of heCAML presence in patients in FIG. 2 with metastatic disease.

FIG. 4 shows the number of heCAMLs in the blood of an initial blood draw from subjects having non-metastatic cancer, single organ metastasis, and multi-organ metastases from n=275 cancer patients.

FIG. 5 shows the number and/or presence of heCAMLs in cancer patients shown in FIG. 4 at various stages.

FIG. 6 shows Kaplan-Meier comparisons of heCAML presence in patients with metastatic disease (A), and the increase in progression and mortality in subjects having CAMLs of different sizes (B).

### DETAILED DESCRIPTION

The matters defined in the description such as a detailed construction and elements are nothing but the ones provided to assist in a comprehensive understanding of the invention. Accordingly, those of ordinary skill in the art will recognize that various changes and modifications of the embodiments described herein can be made without departing from the scope and spirit of the invention.

Cancer is one of the most feared illness in the world, affecting all populations and ethnicities in all countries. Approximately 40% of both men and women will develop cancer in their lifetime. In the United States alone, at any given time there are more than 12 million cancer patients, with 1.7 million new cancer cases and more than 0.6 million deaths estimated for 2018 Cancer death worldwide is estimated to be about 8 million annually, of which 3 million occur in developed countries where patients have access to treatment.

Ideally, there will be diagnostics that can quickly determine if cancer is present and/or whether cancer has metastasized, that can be used to select treatment type, determine whether selected therapy is working, and that can be used to provide prognostics for survival.

In this disclosure, a cell type is presented that is more consistently found in the blood of solid tumor patients from Stages I-IV than any other cancer related cell. These circulating cells are macrophage-like cells that contain the same tumor markers as the primary tumor and they are termed circulating Cancer Associated Macrophage-like cells (CAMLs) herein.

Along with circulating tumor cells (CTCs), CAMLs present in biological samples from patients having cancer can be isolated and characterized, for example through the use of size exclusion methods, including microfiltration methods. Microfilters can be formed with pores large enough to allow red blood cells and the majority of white blood cells to pass, while retaining larger cells such as CTCs and CAMLs. The collected cells can then be characterized, either directly on the filters or through other means.

CAMLs have many clinical utilities when used alone. Furthermore, the characterization of CAMLs in a biological sample can be combined with the assaying of other markers such as CTCs, cell-free DNA and free proteins in blood to further improve sensitivity and specificity of a diagnosis technique. This is especially true for CAMLs and CTCs because they can be isolated and identified at the same time using the same means.

### Circulating Tumor Cells

As defined herein, CTCs associated with carcinomas express a number of cytokeratins (CKs). CK 8, 18, & 19 are the cytokeratins most commonly expressed by CTCs and used in diagnostics, but surveying need not be limited to these markers alone. The surface of solid tumor CTCs usually express epithelial cell adhesion molecule (EpCAM). However, this expression is not uniform or consistent. CTCs do not express any CD45 because it is a white blood cell marker. In assays to identify tumor-associated cells, such as CTCs and CAMLs, it is sufficient to use antibodies against markers associated with the solid tumor such as CK 8, 18, & 19, or antibodies against CD45 or DAPI. Combining staining techniques with morphology, pathologically-definable CTCs (PDCTC), apoptotic CTCs and CAMLs can be identified [3].

PDCTCs associated with carcinoma express CK 8, 18, & 19, and can be identified and defined by the following characteristics:
A “cancer-like” nuclei stained by DAPI. The nuclei are usually large with dot patterns. The exception is when the cell is in division. The nucleus can also be condensed.
Expression of one or more of CK 8, 18 and 19; CTCs from epithelial cancers usually express at least CK 8, 18 and 19. The cytokeratins have a filamentous pattern.
Lack of CD45 expression.

PDCTCs associated with sarcoma express vimentin instead of CK 8, 18, & 19.

PDCTCs associated with melanoma express CD146, CD31 and/or CD34 instead of CK 8, 18, & 19.

PDCTCs associated with neuroblastoma express GD2 and/or vimentin instead of CK 8, 18, & 19.

An apoptotic CTCs associated with carcinoma express CK 8, 18, & 19 and can be identified and defined by the following characteristics:
A degrading nuclei.
Expression of one or more of CK 8, 18 and 19, not all the cytokeratins are filamentous in pattern, but parts or whole appear fragmented in the form of spots.
Lack of CD45 expression.

### Circulating Cancer Associated Macrophage-Like Cells (CAMLs)

As defined herein, the circulating cells used in the methods of the invention can be termed “CAMLs”. Each reference to “circulating cells” is synonymous with CAMLs, and each reference to “CAMLs” is synonymous with circulating cells. Whether termed CAMLs or “circulating cells” these cells are characterized by having one or more of the following features:
CAMLs have a large, atypical polyploid nucleus or multiple individual nuclei, often scattered in the cell, though enlarged fused nucleoli are common. CAML nuclei generally range in size from about 10 μm to about 70 μm in diameter, more commonly from about 14 μm to about 64 μm in diameter.
For many cancers, CAMLs express the cancer marker of the disease. For example, CAMLs associated with epithelial cancers may express CK 8, 18 or 19, vimentin, etc. The markers are typically diffused, or associated with vacuoles and/or ingested material. The staining pattern for any marker is nearly uniformly diffused throughout the whole cell. For sarcomas, neuroblastomas and melanomas, other markers associated with the cancers can be used instead of CK 8, 18, 19.
CAMLs can be CD45 positive or CD45 negative, and the present invention encompasses the use of both types of CAMLs.
CAMLs are large, approximately 20 micron to approximately 300 micron in size by the longest dimension.
CAMLs are found in many distinct morphological shapes, including spindle, tadpole, round, oblong, two legs, more than two legs, thin legs, or amorphous shapes.
CAMLs from carcinomas typically have diffused cytokeratins.
If CAMLs express EpCAM, EpCAM is typically diffused throughout the cell, or associated with vacuoles and/or ingested material, and nearly uniform throughout the whole cell, but not all CAML express EpCAM, because some tumors express very low or no EpCAM.
If CAMLs express a marker, the marker is typically diffused throughout the cell, or associated with vacuoles and/or ingested material, and nearly uniform throughout the whole cell, but not all CAML express the same markers with equal intensity.
CAMLs often express markers associated with the markers of the tumor origin, e.g., if the tumor is of prostate cancer origin and expresses PSMA, then CAMLs from such a patient also expresses PSMA. As another example, if the primary tumor is of pancreatic origin and expresses PDX-1, then CAMLs from such a patient also expresses PDX-1. As further example, if the primary tumor or CTC of the cancer origin express CXCR-4, then CAMLs from such a patient also express CXCR-4.
If the primary tumor or CTC originating from the cancer expresses a biomarker of a drug target, CAMLs from such a patient also express the biomarker of the drug target. An example of such a biomarker of immunotherapy is PD-L1.
CAMLs express monocytic markers (e.g. CD11c, CD14) and endothelial markers (e.g. CD146, CD202b, CD31).
CAMLs have the ability to bind Fc fragments.

CAMLs larger than about 100 μm are termed “hyper-engorged Cancer Associated Macrophage-like cells” or heCAMLs.

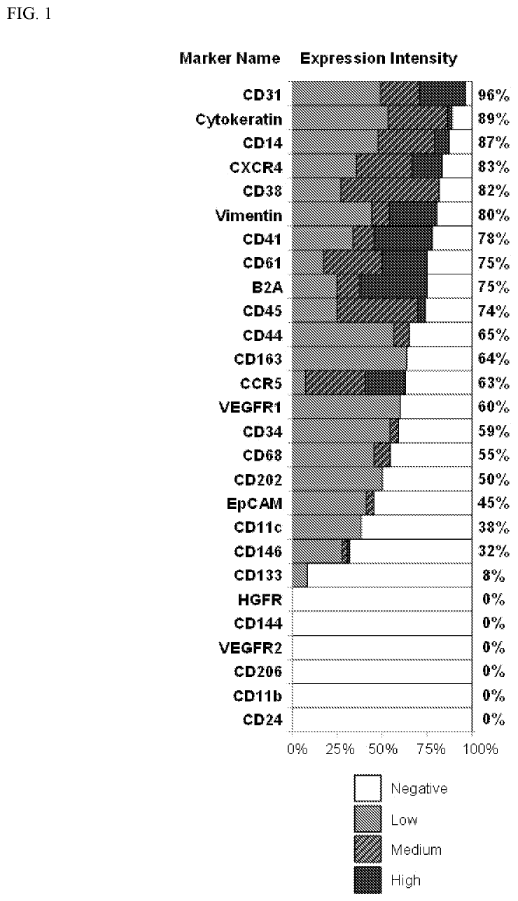

An extensive set of markers were evaluated for their expression on CAMLs, and the results are shown in FIG. 1. In one aspect of the invention, the CAMLs of the present invention express 1, 2, 3, 4, 5, 6, 7, 8, 9, 10, 11, 12, 13, 14, 15, 16, 17, 18, 19, 20 or all 21 of the markers shown in FIG. 1. The markers were screened against 1118 CAMLs from 93 different patients with different cancers CAMLs were initially isolated and identified with DAPI, cytokeratin, and CD45; then sequentially restained with total of 27 markers including myeloid/macrophage, white blood cell, megakaryocyte, epithelial, endothelial, progenitor/stem, and motility markers. As can be seen from FIG. 1, marker expression ranges from 0% to 96%. Almost all CAMLs were found to express levels of CD31, and commonly co-expressed cytokeratin, CD14, CXCR4, vimentin and other markers. However, while CAMLs contained a clear myeloid lineage marker (CD14), CD31 marker was expressed more often at 96%.

CAMLs also present with numerous phenotypes which do not appear to match the understanding of classical cellular differentiation (i.e. co-expression of CD45 [leukocyte] and cytokeratin [epithelial], CD11c [macrophage] and CD41 [megakaryocyte], CD146 [endothelial] and CD41/CD61 [megakaryocyte], CD41/CD61 [megakaryocyte] and CD68/CD163 [scavenger macrophage]). Many of the markers appear on multiple cell types. Combined, these data show CAMLs are myeloid-derived cells early in their differentiation process that possess many phenotypic attributes associated with stem cell and proangiogenic capabilities.

CAMLs can be visualized by colorimetric stains, such as H&E, or fluorescent staining of specific markers as shown in FIG. 1. For the cytoplasm, CD31 is the most positive phenotype. CD31 alone, or in combination with other positive markers in FIG. 1, or cancer markers associated with the tumor are recommended.

In the various embodiments and aspects of the invention, CAMLs can be defined as cells having each of the following characteristics: (a) a large atypical polyploid nucleus of about 14-64 μm in size, or multiple nuclei in a single cell; (b) cell size of about 20-300 microns in size; and (c) a morphological shape selected from the group consisting of spindle, tadpole, round, oblong, two legs, more than two legs, thin legs, and amorphous. In further embodiments, the CAMLs can be defined as also having one or more of the following additional characteristics: (d) CD14 positive phenotype; (e) CD45 expression; (f) EpCAM expression; (g) vimentin expression; (h) PD-L1 expression; (i) monocytic CD11C marker expression; (j) endothelial CD146 marker expression; (k) endothelial CD202b marker expression; and (l) endothelial CD31 marker expression.

As suggested above, the unique characteristics of the CAMLs and CTCs described herein make them well-suited for use in clinical methodology including methods of screening and diagnosis diseases such as cancer, monitoring treatment, monitoring of disease progression and recurrence.

### Predicting and Diagnosing Multiple Organ Metastasis

As suggested in the Summary above, the present invention is directed to methods for predicting development of or diagnosing multiple organ metastasis and/or multifocal metastatic disease in a subject. The methods comprise determining the size of CAMLs in a biological sample from a subject, such as a subject having cancer, wherein when at least one CAML in said sample is about 100 μm or more in size, the subject is predicted to develop or diagnosed to have multiple organ metastases and/or multifocal metastatic disease.

### Predicting OS and PFS Using Cell Size

The present invention is also directed to methods for predicting overall survival (OS) and/or progression free survival (PFS) of a subject having cancer based on CAML cell size. The methods comprise determining the size of CAMLs in a biological sample from a subject, such as a subject having cancer, wherein when at least one CAML in said sample is about 100 μm or more in size, the subject is predicted to have shorter OS and/or shorter PFS than a subject having the same or similar cancer but lacking at least one CAML in a corresponding sample about 100 μm or more in size. Thus, the OS and/or PFS of a subject with larger-sized CAMLs is predicted to be less or shorter than the OS and/or PFS of a subject having smaller-sized CAMLs. In certain aspects, the cancer is multiple organ metastasis or multifocal metastatic disease.

In preferred aspects of the invention, the methods comprise determining the size of CAMLs in a biological sample from a subject having cancer, wherein when at least one CAML in said sample is about 100 μm or more in size, the OS and PFS of the subject is predicted to be less or shorter than a subject having cancer where none of the CAMLs is more than about 100 μm in size.

The 100 μm value can be considered a cut-off value for this method. In related aspects of this method, the cut-off value may be any one of 90, 91, 92, 93, 94, 95, 96, 97, 98, 99, 100, 101, 102, 103, 104, 105, 106, 107, 108, 109, or 110 μm or more.

In one aspect of the invention, the methods of the invention comprise determining the size of CAMLs in a biological sample from a subject having cancer, wherein when at least one cell in said sample is about 100 μm or more in size, the OS and/or PFS of the subject is predicted to be less or shorter than a subject having cancer where none of the cells is more than about 100 μm in size.

With respect to this method, it is important to recognize that the size of a particular CAML can vary depending on the condition and morphology of the cell, the circumstances under which the size is determined, and the manner in which the size is determined. Because the types of morphologies adopted by the circulating cells include spindle, tadpole, round, oblong, two legs, etc. (as further defined herein), the size can also vary depending on the two points on a cell selected for measurement. However, the size of the cell will generally be measured between the two points most distant on the body of the cell. Thus, if the shape is round, the diameter of the cell is measured. If the shape is spindle, the distance between the two ends along the axial length of the cell can be measured.

In each of the methods of the invention, overall survival (OS) or progression free survival (PFS), or both, is over a period of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 months, or more. In one aspect of the invention, OS or PFS, or both, is over a period of at least about 12 months or over a period of at least about 24 months.

As used herein, overall survival (OS) means the length of time survived by a subject having cancer from a selected date, such as the date of diagnosis, the date on which treatment began, and the date on which blood is drawn to assess cancer progression.

As used herein, progression free survival (PFS) means the length of time survived by a subject having cancer from a selected date, such as the date on which treatment began or the date on which blood is drawn to assess cancer progression, and where the cancer has not worsened or progressed.

In each of the methods of the invention, it will be apparent that the amount of the biological sample in which the circulating cells (CAMLs) are assayed can vary. However, to obtain a relevant number of cells when the methods are based on determining the size of the cells, the biological sample should be at least about 2.5 mL. The amount of biological sample may also be at least about 3, 4, 5, 6, 7, 7.5, 8, 9, 10, 11, 12, 12.5, 13, 14, 15, 16, 17, 17.5, 18, 19, 20, 21, 22, 22.5, 23, 24, 25, 26, 27, 27.5, 28, 29, or 30 mL, or more. The amount of biological sample may also be between about 2.5 and 20 mL, between about 5 and 15 mL, or between about 5 and 10 mL. In one aspect of the invention, the biological sample is about 7.5 mL.

In each of the embodiments and aspects of the invention, the circulating cells (CAMLs) can be defined as having each of the following characteristics:
(a) large atypical polyploid nucleus of about 14-64 μm in size, or multiple nuclei in the same cell;
(b) cell size of about 20-300 μm in size; and
(c) morphological shape selected from the group consisting of spindle, tadpole, round, oblong, two legs, more than two legs, thin legs, and amorphous.

In certain aspects of the embodiments of the invention, the circulating cells (CAMLs) can be further defined as having one or more of the following additional characteristics.
(d) CD14 positive phenotype;
(e) CD45 expression;
(f) EpCAM expression;
(g) vimentin expression;
(h) PD-L1 expression;
(i) monocytic CD11C marker expression;
(j) endothelial CD146 marker expression;
(k) endothelial CD202b marker expression; and
(l) endothelial CD31 marker expression.

In each of the embodiments and aspects of the invention, the source of the biological sample may be, but is not limited to, one or more of peripheral blood, blood, lymph node, bone marrow, cerebral spinal fluid, tissue, and urine. When the biological sample is blood, the blood may be antecubital-vein blood, inferior-vena-cava blood femoral vein blood, portal vein blood, or jugular-vein blood, for example. The sample may be a fresh sample or a cryo-preserved sample that is thawed [8].

When comparing the circulating cells (CAML) sizes between two subjects having cancer, it is preferable for the subjects to have the same type of cancer. However, it may be difficult to fully match two subjects in terms of the type of cancer, the stage of the cancer, the rate of progression of the cancer, history of treatment, and history of remission and/or reoccurrence of the cancer, among other factors. Therefore, it should be understood that there may be some variations in the characteristics in the cancers of two subject that are being compared in the methods of the invention.

In each of the embodiments and aspects of the invention, the cancer is a solid tumor, Stage I cancer, Stage II cancer, Stage III cancer, Stage IV cancer, carcinoma, sarcoma, neuroblastoma, melanoma, epithelial cell cancer, breast cancer, prostate cancer, lung cancer, pancreatic cancer, colorectal cancer, renal cancer, liver cancer, head and neck cancer, kidney cancer, ovarian cancer, esophageal cancer or other solid tumor cancer. The skilled artisan will appreciate that the methods of the invention are not limited to particular forms or types of cancer and that they may be practiced in association with a wide variety of cancers.

In each of the embodiments and aspects of the invention, the circulating cells (CAMLs) are isolated from the biological samples for the determining steps using one or more means selected from size exclusion methodology, immunocapture, red blood cell lysis, white blood cell depletion, FICOLL, electrophoresis, dielectrophoresis, flow cytometry, magnetic levitation, and various microfluidic chips, or a combination thereof. In a particular aspect, the size exclusion methodology comprises use of a microfilter.

In one aspect of the invention, circulating cells (CAMLs) are isolated from the biological samples using size exclusion methodology that comprises using a microfilter. Suitable microfilters can have a variety of pore sizes and shapes. The microfilter may have a pore size ranging from about 5 microns to about 20 microns. In certain aspects of the invention, the pore size is between about 5 and 10 microns; in other aspects, the pore size is between about 7 and 8 microns. The larger pore sizes will eliminate most of the WBC contamination on the filter. The pores of the microfilter may have a round, race-track shape, oval, square and/or rectangular pore shape. The microfilter may have precision pore geometry and/or uniform pore distribution.

In another aspect of the invention, circulating cells (CAMLs) are isolated from the biological samples using a microfluidic chip via physical size-based sorting, hydrodynamic size-based sorting, grouping, trapping, immunocapture, concentrating large cells, or eliminating small cells based on size. The circulating cell (CAML) capture efficiency can vary depending on the collection method. The circulating cell (CAML) size that can be captured on different platforms can also vary. The principle of using circulating cell size to determine prognosis and survival is the same, but the statistics will vary. Collection of circulating cells using CellSieve™ microfilters provides 100% capture efficiency and high quality cells.

In another aspect of the invention is the blood collection tube. CellSave blood collection tubes (Menarini Silicon Biosystems Inc., San Diego, CA) provide stable cell morphology and size. Other available blood collection tubes do not provide cell stability. Cells can enlarge and may even burst collect in most other blood collection tubes.

Another aspect of the invention is to identify large cells in the sample without specifically identifying the cells as CAMLs per se, instead simply identifying the cells based on size of the cytoplasm and nucleus. Examples are techniques that may be used in this aspect include using color metric stains, such as H&E stains, or just looking at CK (+) cells.

In a further aspect of the invention, circulating cells (CAMLs) are isolated from the biological samples using a Creatv MicroTech CellSieve™ low-pressure microfiltration assay. Size-exclusion methods are ideal for isolating CAMLs from the blood stream. Creatv MicroTech's CellSieve™ microfilter is a size-exclusion device with 7.5 μm-diameter pores, 180,000 pores distributed uniformly within a 9 mm-diameter area on a strong, low-autofluorescence, 10 μm-thick polymer. Filtration by size is a suitable method to consistently capture multiple types of tumor-associated cells in the blood, both CTCs and CAMLs. Filtration can be performed under low pressure using a syringe pump or a vacuum pump.

For example, whole blood is collected in a CellSave Preservative Tube. 7.5 mL of whole blood is prefixed in 7.5 mL of prefixation buffer. The 15 mL sample is passed through the CellSieve™ microfilter in 3 min. The microfilter removes all red blood cells and 99.9% of white blood cells. The assay is followed by fixation, permeabilization, and fluorescence staining of the cells captured on the filter. The microfilter is then mounted on a glass slide and imaged on a fluorescent microscope

### EXPERIMENTS

### Experiment #1

Methods. 151 patients were prospectively recruited with metastatic (m) mBreast (n=58), mLung (n=34), mProstate (n=39), and mRenal (n=20) cancers. Peripheral blood was collected prior to the induction of new treatment for metastatic cancer. CAMLs were isolated following standard CellSieve techniques from 7.5 mL of blood, then imaged/measured using ZenBlue. Multi-organ metastasis was defined as spread to ≥2 distant organ sites, or any spread to the brain. Single factor analysis of variance (ANOVA) was used to compare heCAML presence in multi-organ metastases versus patients with single organ metastasis. Univariate and multivariate analysis was run to evaluate PFS and OS against heCAMLs, and all known clinical parameters.

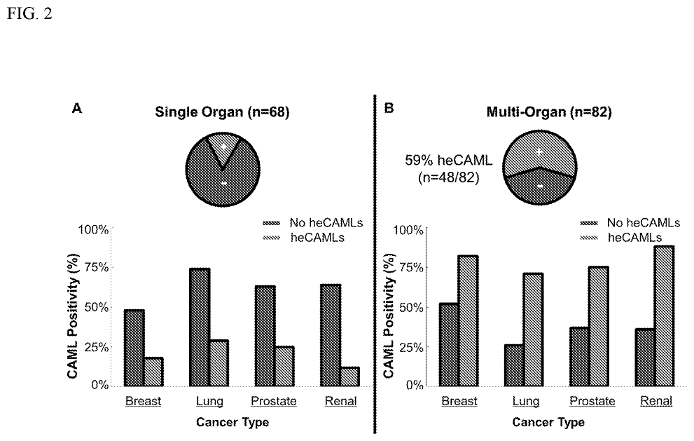

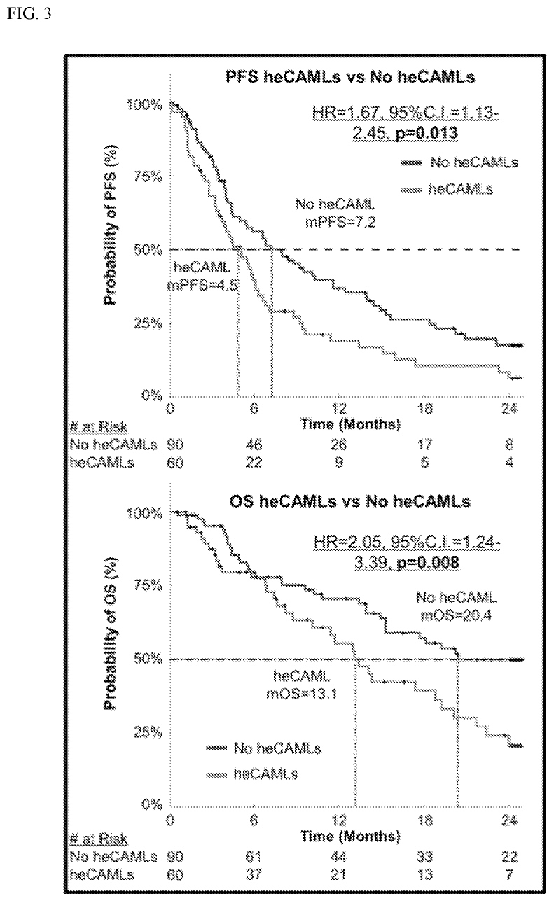

Results. Multi-organ metastases were present in 55% (n=83/150) of the patients (Table 1). heCAMLs were found in 59% (n=49/83) of patients with multi-organ metastases (FIG. 2B), while heCAMLs were found in only 16% (n=11/67) of patients with single organ metastasis (FIG. 2A). heCAML presence appeared to indicate multi-organ metastasis in mBreast (82% vs. 52%, p=0.006), mLung (71% vs. 26%, p=0.025), mProstate (75% vs. 37%, p=0,029), and mRenal (88% vs. 36%, p=0.025) cancer patients (FIG. 2B). Further, in all n=150 patients, heCAML presence predicted a significantly shorter median PFS of 4.5 versus 7.2 months, 24 month PFS (HR=1.67, 95% CI=1.13-2.45, p=0.013), and significantly shorter median OS of 13.1 versus 20.4 months, 24 month OS (HR=2.05, 95% CI=1.24-3.39, p=0.008) (FIG. 3).

TABLE 1
Patient Demographic Table

| | Breast | | Lung | | Prostate | | Renal Cell | |
|---|---|---|---|---|---|---|---|---|
| Demographic | (n = 58) | | (n = 34) | | (n = 39) | | (n = 20)* | |
| Age(Median): [Range] | 57 | [27-92] | 62.5 | [45-81] | 73 | [48-89] | 61 | [42-78] |

Type of Metastasis

| | | | | | | | | |
|---|---|---|---|---|---|---|---|---|
| Single Organ | 18 | (31%) | 22 | (65%) | 20 | (51%) | 8 | (40%) |
| Multi-Organ | 40 | (69%) | 12 | (35%) | 19 | (49%) | 12 | (60%) |

Metastasis Location

| | | | | | | | | |
|---|---|---|---|---|---|---|---|---|
| Bone | 34 | (59%) | 10 | (30%) | 34 | (87%) | 6 | (30%) |
| Brain | 9 | (16%) | 9 | (27%) | 0 | (0%) | 4 | (20%) |
| Liver | 27 | (47%) | 5 | (15%) | 5 | (13%) | 5 | (25%) |
| Local | 5 | (9%) | 7 | (21%) | 1 | (3%) | 0 | (0%) |
| Lung | 15 | (26%) | 7 | (21%) | 6 | (18%) | 18 | (90%) |
| Other | 16 | (28%) | 4 | (12%) | 13 | (33%) | 6 | (30% |

heCAML Presence

| | | | | | | | | |
|---|---|---|---|---|---|---|---|---|
| Absence | 25 | (43%) | 27 | (79%) | 27 | (69%) | 11 | (58%) |
| Presence | 33 | (57%) | 7 | (21%) | 12 | (31%) | 8 | (42%) |

heCAML Presence Single Organ

| | | | | | | | | |
|---|---|---|---|---|---|---|---|---|
| Absence | 12 | (67%) | 20 | (91%) | 17 | (85%) | 7 | (87%) |
| Presence | 6 | (33%) | 2 | (9%) | 3 | (15%) | 1 | (13%) |

heCAML Presence Multi-Organ

| | | | | | | | | |
|---|---|---|---|---|---|---|---|---|
| Absence | 13 | (32%) | 7 | (58%) | 10 | (53%) | 4 | (36%) |
| Presence | 27 | (68%) | 5 | (42%) | 9 | (47%) | 7 | (64%) |

(*Excludes 1 failed sample)

Conclusions. From a single blood draw, patients with at least one heCAMLs were identified as more likely to have multi-organ metastases. heCAML presence predicted for significantly shorter PFS (HR=1.67) and OS (HR=2.05). heCAML presence prior to initiation of new treatment may predict for multi-organ metastases, thus requiring more aggressive treatment regimes.

### Experiment #2

Methods. 275 patients were prospectively recruited with breast cancer (n=58), lung cancer (n=47), prostate cancer (n=47), pancreatic cancer (n=29), esophageal cancer (n=28), renal cancer (n=20), colorectal cancer (n=15), liver cancer (n=9), and sarcoma (n=22), including Stage I (n=19), Stage II (n=31), Stage III (n=65), and Stage IV (n=160). Peripheral blood was collected prior to the induction of new treatment. Cells were isolated following standard CellSieve™ techniques from 7.5 mL of blood, then imaged and measured in ZenBlue. Multi organ metastasis was defined as spread to ≥2 distant organ sites, or any spread to the brain. Single factor ANOVA was conducted to compare heCAML presence in multi organ metastatic patients versus patients with single organ site metastasis. Univariate and multivariate analysis was run to evaluate for PFS and OS against heCAMLs, and all known clinical parameters.

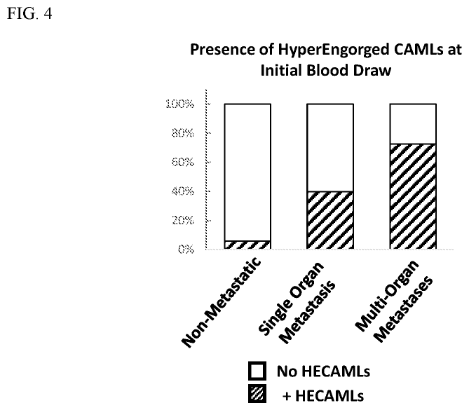

Results. 275 viable samples were obtained. Multi organ metastases were present in 57% (n=91/160) of patients with metastatic disease at initial blood draw (Table 2). heCAMLs were found in 73% (n=66/91) of the initial multi organ metastatic population, but only in 19% (n=22/115) of the non-metastatic cohort (p<0.001) (FIG. 4), thus HeCAMLs are common to multi-organ metastases and single organ metastasis and rare in clinically diagnosed non-metastatic disease.

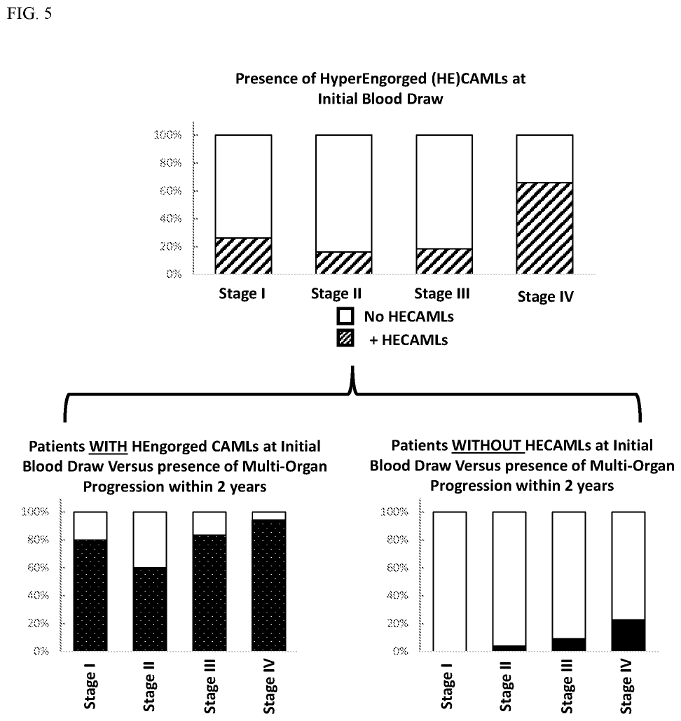

Of the n=22/115 non-metastatic patients with heCAML present at initial blood draw, 73% (n=16/22) progressed with multi organ metastases within 2 years of blood draw (FIG. 5). HeCAMLs were identified in all stages in a variety of solid cancer types, including sarcoma, and staged by standard clinical or pathological methodology. Within 2 years, patients that had HeCAMLs at initial staging were highly likely to progress with multi-organ metastases, including 80% of stage I, 60% of Stage II, 83% of Stage III and 94% of Stage IV patients. In contrast, within 2 years, patients that did not have HeCAMLs at initial staging were unlikely to progress with multi-organ metastases, including 0% of stage 1, 4% of Stage II, 9% of Stage III and 23% of Stage IV patients.

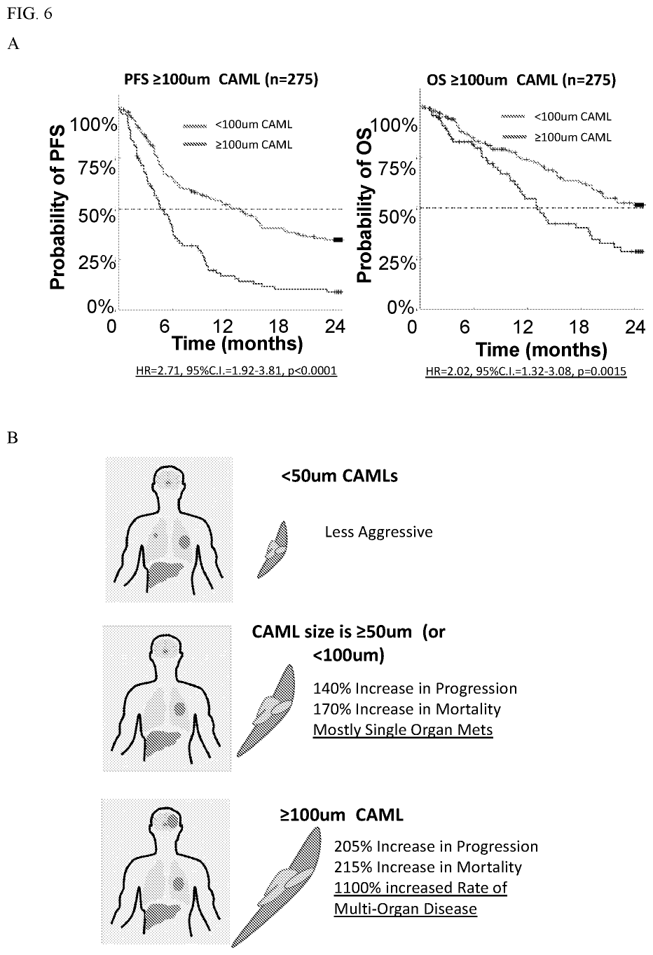

In all n=275 patients, heCAML presence predicted a significantly shorter 24 month PFS (HR=2.71, 95% CI=1.92-3.81, p<0.0001), and significantly shorter 24 month OS (HR=2.02, 95% CI=1.32-3.08, p=0.0015) (FIG. 6A) FIG. 6B shows overview summary of n=275 patients with solid carcinomas from Table 2. <50 μm CAMLs appear to have less aggressive disease, less likely to progress or die. Patients with ≥50 μm to <100 μm CAMLs have a 140% increased risk of progression and a 170% increased risk of death within 2 years, with most patients having only single organ metastasis. Patients with ≥100 μm CAMLs have a 205% increased risk of progression and 1100% increased risk of death within 2 years, with most patients having multi-organ metastases.

Conclusions. A non-invasive prognostic blood based assay was developed and examined to determine its relationship to multi organ metastatic spread as well as its prognostic value in several solid cancers. These results showed patients with heCAMLs had higher rates of multi organ metastases, and appear to predict for shorter PFS and OS.

TABLE 2

| | | | | | | | | | | |
|---|---|---|---|---|---|---|---|---|---|---|
| | Total | Breast | Lung | Prostate | Pancreas | Esophageal | Sarcoma | Renal Cell | Colon | Liver |
| Demographic | (n = 275) | (n = 58) | (n = 47) | (n = 47) | (n = 29) | (n = 28) | (n = 22) | (n = 20) | (n = 15) | (n = 9) |

Type of Metastasis

| | | | | | | | | | | |
|---|---|---|---|---|---|---|---|---|---|---|
| None | 92 | 7 | 33 | 8 | 26 | 24 | 4 | 0 | 2 | 8 |
| Single Organ | 69 | 13 | 11 | 20 | 3 | 0 | 6 | 8 | 6 | 1 |
| Multi-Organ | 114 | 38 | 2 | 19 | 0 | 0 | 12 | 12 | 7 | 0 |

heCAML Presence

| | | | | | | | | | | |
|---|---|---|---|---|---|---|---|---|---|---|
| Absence | 92 | 25 | 36 | 35 | 21 | 22 | 11 | 11 | 9 | 9 |
| Presence | 183 | 33 | 11 | 12 | 8 | 2 | 11 | 9 | 6 | 0 |

heCAML Presence Single Organ

| | | | | | | | | | | |
|---|---|---|---|---|---|---|---|---|---|---|
| Absence | 64 | 20 | 0 | 19 | 1 | N/A | 6 | 7 | 7 | 1 |
| Presence | 4 | 4 | 12 | 1 | 2 | N/A | 0 | 1 | 0 | 0 |

heCAML Presence Multi-Organ

| | | | | | | | | | | |
|---|---|---|---|---|---|---|---|---|---|---|
| Absence | 66 | 9 | 2 | 8 | 0 | N/A | 1 | 4 | 0 | N/A |
| Presence | 87 | 29 | 0 | 11 | 0 | N/A | 11 | 8 | 6 | N/A |

### CITATIONS

1. Adams, D., et al., Circulating giant macrophages as a potential biomarker of solid tumors. PNAS 2014, 111(9):3514-3519.
2. International Patent Application Publication No. WO 2013/181532, dated Dec. 5, 2013.
3. Adams, D. L., et al., Cytometric characterization of Circulating Tumor Cells captured by microfiltration and their correlation to the CellSearch® CTC test. Cytometry Part A 2015; 87A:137-144.
4. Adams D L, et al. The systematic study of circulating tumor cell isolation using lithographic microfilters. RSC Adv 2014, 4:4334-4342.
5. Adams et al., Multi-phenotypic subtyping of circulating tumor cells using sequential fluorescent quenching and restaining, Scientific Reports 2016, 6:33488|DOI: 10.1038/srep33488.
6. International Patent Application Publication No. WO 2013/078409, dated May 30, 2013.
7. International Patent Application Publication No. WO 2016/33103, dated Mar. 3, 2016.
8. Zhu P, et al., Detection of Tumor-Associated Cells in Cryopreserved Peripheral Blood Mononuclear Cell Samples for Retrospective Analysis, J of Translational Medicine, 2016, 14:198. DOI: 10.1186/s12967-016-0953-2.
9. Adams, D. et al. Cancer-Associated Macrophage-Like Cells as Prognostic Indicators of Overall Survival in a Variety of Solid Malignancies. J. Clin. Onco. 35, 11503 (2017).
10. Steeg, P. et al. Tumor Metastasis: Mechanistic Insights and Clinical Challenges. Nature Medicine. 12 (2006).
11. Valastyan, S. et al. Tumor Metastasis: Molecular Insights and Evolving Paradigms. Cell 147 (2011).

1. A method for diagnosing multiple organ metastasis and/or multifocal metastatic disease in a subject, said method comprising determining the size of CAMLs in a biological sample from a subject, such as a subject having cancer, wherein when at least one CAML in said sample is about 100 μm or more in size, the subject is diagnosed to have multiple organ metastases and/or multifocal metastatic disease.

2. A method for predicting development of multiple organ metastasis and/or multifocal metastatic disease in a subject, said method comprising determining the size of CAMLs in a biological sample from a subject, such as a subject having cancer, wherein when at least one CAML in said sample is about 100 μm or more in size, the subject is predicted to develop multiple organ metastases and/or multifocal metastatic disease.

3. A method for predicting overall survival (OS) and/or progression free survival (PFS) of a subject having cancer, said method comprising determining the size of CAMLs in a biological sample from a subject having cancer, wherein when at least one CAML in said sample is about 100 μm or more in size, the subject is predicted to have shorter OS and/or shorter PFS than a subject having the same or similar cancer but lacking at least one CAML in a corresponding sample about 100 μm or more in size.

4. A method for predicting OS and/or PFS of a subject having cancer, said method comprising determining the size of CAMLs in a biological sample from a subject having cancer, wherein when at least one CAML in said sample is about 100 μm or more in size, the OS and/or PFS of the subject is predicted to be less or shorter than a subject having cancer where none of the CAMLs is more than about 100 μm in size.

5. The method of claim 3, wherein said OS and/or PFS is over a period of at least 12 months.

6. The method of claim 3, wherein said OS and/or PFS is over a period of at least 24 months.

7. The method of claim 1, wherein the size of the biological sample is between 5 and 15 mL.

8. The method of claim 1, wherein the CAMLs have the following characteristics:
(a) large atypical polyploid nucleus of about 14-64 μm in size, or multiple nuclei in a single cell;
(b) cell size of about 20-300 μm in size; and
(c) morphological shape selected from the group consisting of spindle, tadpole, round, oblong, two legs, more than two legs, thin legs, and amorphous.

9. The method of claim 8, wherein the CAMLs have one or more of the following additional characteristics:
(d) CD14 positive phenotype;
(e) CD45 expression;
(f) EpCAM expression;
(g) vimentin expression;
(h) PD-L1 expression;
(i) monocytic CD11C marker expression;
(j) endothelial CD146 marker expression;
(k) endothelial CD202b marker expression; and
(l) endothelial CD31 marker expression.

10. The method of claim 1, wherein the source of the biological sample is one or more of peripheral blood, blood, lymph node, bone marrow, cerebral spinal fluid, tissue, and urine.

11. The method of claim 10, wherein the biological sample is antecubital-vein blood, inferior-vena-cava blood, femoral vein blood, portal vein blood, or jugular-vein blood.

12. The method of claim 1, wherein the cancer is a solid tumor, Stage I cancer, Stage II cancer, Stage III cancer, Stage IV cancer, carcinoma, sarcoma, neuroblastoma, melanoma, epithelial cell cancer, breast cancer, prostate cancer, lung cancer, pancreatic cancer, colorectal cancer, renal cancer, liver cancer, head and neck cancer, kidney cancer, ovarian cancer, esophageal cancer or other solid tumor cancer.

13. The method of claim 1, wherein CAMLs are isolated from the biological samples for the determining steps using one or more means selected from the group consisting of size exclusion methodology, immunocapture, red blood cell lysis, white blood cell depletion, FICOLL, electrophoresis, dielectrophoresis, flow cytometry, magnetic levitation, and various microfluidic chips, or a combination thereof.

14. The method of claim 13, wherein CAMLs are isolated from the biological samples using size exclusion methodology that comprises using a microfilter.

15. The method of claim 14, wherein the microfilter has a pore size ranging from about 5 microns to about 20 microns.

16. The method of claim 15, wherein the pores of the microfilter have a round, race-track shape, oval, square and rectangular pore shape.

17. The method of claim 15, wherein the microfilter has precision pore geometry and uniform pore distribution.

18. The method of claim 13, wherein CAMLs are isolated using a microfluidic chip via physical size-based sorting, hydrodynamic size-based sorting, grouping, trapping, immunocapture, concentrating large cells, or eliminating small cells based on size.

19. The method of claim 1, wherein CAMLs are isolated from the biological samples for the determining steps using a low-pressure microfiltration assay.
